(19) United States
(12) Patent Application Publication
(10) Pub. No.: US 2013/0144130 A1
(43) Pub. Date: Jun. 6, 2013

(54) SYSTEM METHOD AND DEVICE FOR MONITORING A PERSON'S VITAL SIGNS

(71) Applicants: Brian K. Russell, Crownsville, CT (US); Jonathan Woodward, Annapolis, MD (US)

(72) Inventors: Brian K. Russell, Crownsville, CT (US); Jonathan Woodward, Annapolis, MD (US)

(73) Assignee: ZEPHYR TECHNOLOGY CORPORATION, Annapolis, MD (US)

(21) Appl. No.: 13/361,633

(22) Filed: Jan. 30, 2012

Related U.S. Application Data
(60) Provisional application No. 61/438,298, filed on Feb. 1, 2011.

(51) Int. Cl. A61B 5/0205

(52) U.S. Cl. CPC A61B 5/0205
USPC 600/301

(57) ABSTRACT

Provided is a system, method and device for determining one or more physiological parameters of a person.

### CROSS-REFERENCE TO RELATED APPLICATIONS

This application claims priority to U.S. Provisional Patent Application Ser. No. 61/438,298, filed 1 Feb. 2011, the complete disclosure of which is incorporated herein by reference.

### FIELD OF THE INVENTION

The present invention generally relates to physiological data processing and more particularly, to a system, method and device for determining one or more physiological parameters of a person.

### BACKGROUND OF THE INVENTION

Monitoring vital signs is traditionally done on supine patients at rest. Field based measurements are typically done with a care giver or researcher controlling the person's position (e.g., posture) and degree of movement in order to minimise movement artefacts such as orthstatic changes and effects on the body due to work effort of orientation. Normally tests are performed under various conditions in a clinic manually, using such devices as blood pressure cuffs, electrocardiogram (ECG) devices, face masks and using treadmills for exertion tests.

Measuring vital signs over time (in the field) provides more useful information to allow an understanding of a person's physiological state. However, body activity level may affect a person's vital signs and hence the interpretation thereof.

An ECG measures the electrical activity of a person's heart over time captured by electrodes attached to the person's skin. The ECG waveform data, however, may be adversely impacted due to the activity level (movement) of the person, noise, environmental factors, posture, and/or other factors. For example, movement of a person wearing the skin electrodes connected to an ECG device may cause the ECG waveform data to be nearly unusable. Thus, a system for monitoring a person's heart that considers the movement of the person, environmental factors, posture of the person, and signal noise is needed.

These and other advantages may be provided by one or more embodiments of the present invention.

### SUMMARY OF THE INVENTION

The above objectives and other objectives are obtained by a method of monitoring the heart of a person, comprising:
collecting a plurality of sets of ECG waveform data of the person;
wherein each set of ECG waveform data includes a Q portion, an R portion, an S portion, and a T portion for each heart beat;
storing the ECG waveform data in a memory;
concurrently with said collecting a plurality of sets of ECG waveform data of the person, collecting data of an activity level of the person;
storing data the activity level in a memory;
for each of the plurality of sets of ECG waveform data, determining whether the activity level of the person exceeds a threshold during collection of the set of ECG waveform data;
outputting the set of ECG waveform data if the activity level of the person does not exceed the threshold during collection of the set of ECG waveform data; and
if the activity level of the person during collection of the set of ECG waveform data exceeds the threshold, filtering the ECG waveform data to output data of the R portion of the ECG waveform data for each heart beat of the set of ECG waveform data and not output data of the Q portion, S portion, or T portion for each heart beat of the set of data.

The objectives are further obtained by a method of monitoring the heart of a person, comprising:
collecting a plurality of sets of ECG waveform data of the person;
wherein each set of ECG waveform data includes a Q portion, an R portion, an S portion, and a T portion for each heart beat;
concurrently with said collecting a plurality of sets of ECG waveform data of the person, collecting data of an activity level of the person;
for each of the plurality of sets of ECG waveform data, determining whether the activity level of the person during collection of a set of ECG waveform data exceeds a threshold;
outputting the set of ECG waveform data if the activity level of the person during collection of the set of ECG waveform data does not exceed the threshold; and
if the activity level of the person during collection of the set of ECG waveform data exceeds the threshold, filtering the ECG waveform data to exclude the Q portion, S portion, and T portion for each heart beat of the set of data and not excluding the R portion for each heart beat of the set of data; and
outputting the filtered ECG waveform data.

The objectives are also obtained by a computer program product, comprising a computer usable medium having a computer readable program code embodied therein, said computer readable program code adapted to be executed to implement a method for monitoring the heart of a person, the method comprising:
receiving a plurality of sets of ECG waveform data of the person; wherein each set of ECG waveform data includes a Q portion, an R portion, an S portion, and a T portion for each heart beat;
storing the ECG waveform data in a memory;
receiving data of an activity level of the person collected concurrently with the collection the plurality of sets of ECG waveform data of the person;
storing data the activity level in a memory;
for each of the plurality of sets of ECG waveform data, determining whether the activity level of the person during collection of a set of ECG waveform data exceeds a threshold;
outputting the set of ECG waveform data if the activity level of the person during collection of the set of ECG waveform data does not exceed the threshold; and
if the activity level of the person during collection of the set of ECG waveform data exceeds the threshold, filtering the ECG waveform data to exclude the Q portion, S portion, and T portion for each heart beat of the set of data and not excluding the R portion for each heart beat of the set of data; and
outputting the filtered ECG waveform data

The objectives are further obtained by a method of monitoring a vital sign of a person, comprising:
collecting a plurality of sets of physiological waveform data of the person;
for each of the plurality of sets of physiological waveform data, determining whether a trigger condition is satisfied during collection of a set of physiological waveform data;
outputting the set of physiological waveform data if the trigger condition is not satisfied during collection of the set of physiological waveform data; and
if the trigger condition is satisfied during collection of the set of ECG waveform data, filtering the physiological waveform data and outputting the filtered physiological waveform data.

The objectives are also obtained by a method of monitoring a vital sign of a person, comprising:
collecting a plurality of sets of physiological waveform data of the person;
for each of the plurality of sets of physiological waveform data, determining whether a trigger condition is satisfied during collection of a set of physiological waveform data;
outputting the set of physiological waveform data if the trigger condition is not satisfied during collection of the set of physiological waveform data; and
if the trigger condition is satisfied during collection of the set of physiological waveform data, performing the steps of:
determining whether an interfering signal can be extracted from the set of physiological waveform data and if so, extracting the interfering signal from the physiological waveform data and outputting the physiological waveform data with the interfering signal extracted;
if the interfering signal cannot be extracted from the physiological waveform data, filtering the physiological waveform data and outputting the filtered physiological waveform data.

### BRIEF DESCRIPTION OF THE DRAWINGS

The invention is further described in the detailed description that follows, by reference to the noted drawings by way of non-limiting illustrative embodiments of the invention, in which like reference numerals represent similar parts throughout the drawings. As should be understood, however, the invention is not limited to the precise arrangements and instrumentalities shown. In the drawings:

FIG. 1 depicts an example ECG waveform for a single heart beat.

FIG. 2 depicts filtering according to an example embodiment of the present invention.

FIG. 3 is a flow chart of a process, in accordance with an example embodiment of the present invention.

FIG. 4 depicts a BioHarness that may be used to collect (and process data), in accordance with an example embodiment of the present invention.

FIG. 5 illustrates an ECG waveform output including filtered and unfiltered portions according to an example embodiment of the present invention.

FIG. 6 is a flow chart of a process, in accordance with another example embodiment of the present invention.

FIG. 7 provides a functional block diagram of an example embodiment of the present invention.

FIG. 8 is a flow chart of a process, in accordance with another example embodiment of the present invention.

### DETAILED DESCRIPTION OF ILLUSTRATIVE EMBODIMENTS

In the following description, for purposes of explanation and not limitation, specific details are set forth, such as particular networks, communication systems, computers, terminals, devices, components, techniques, data and network protocols, software products and systems, operating systems, development interfaces, hardware, etc. in order to provide a thorough understanding of the present invention.

However, it will be apparent to one skilled in the art that the present invention may be practiced in other embodiments that depart from these specific details. Detailed descriptions of well-known networks, communication systems, computers, terminals, devices, components, techniques, data and network protocols, software products and systems, operating systems, development interfaces, and hardware are omitted so as not to obscure the description.

Embodiments of the present invention address the issue of monitoring a person's vital signs in the field (e.g., at home, in a gym, at work, etc.) and while the person is engaging in any activity, which may include running, walking, jumping, and/or playing sports (e.g., basketball, football, tennis, racquetball, baseball, etc.). The present invention provides a novel way to derive valid vital sign data such as heart rate data from ECG waveform data and breathing rate data collected during various activity levels and/or under other conditions using a combination of biomechanical sensors, physiological sensors and algorithms that process the data over time.

Thus, example embodiments of the present invention generally relate to physiological data processing and more particularly, to a system, method and device for determining one or more physiological parameters of a person and decoupling (removing) movement based artifacts by changing the frequency components analysed and/or by performing time or frequency domain subtraction of such components resulting in the desired physiological vital sign. Vital sign measurements such as heart and breathing waveforms can be disturbed by movement artifacts. Movement of the person can create various interfering signals from lead movement, sensor to skin impedance changes, tissue ionic disturbances and un wanted tissue electrical signals. In some embodiments, these interfering signals may be removed from the desired signal (e.g., heart rate and/or breathing rate) by adapting the frequency used to collected the desired signals, such as by reducing the bandwidth of an input filter, employing one or more notch filters, and/or performing phase analyses. Additionally the interfering signal can be analyzed and used with the total signal to determine the desired physiological vital sign signal (without the interfering signal).

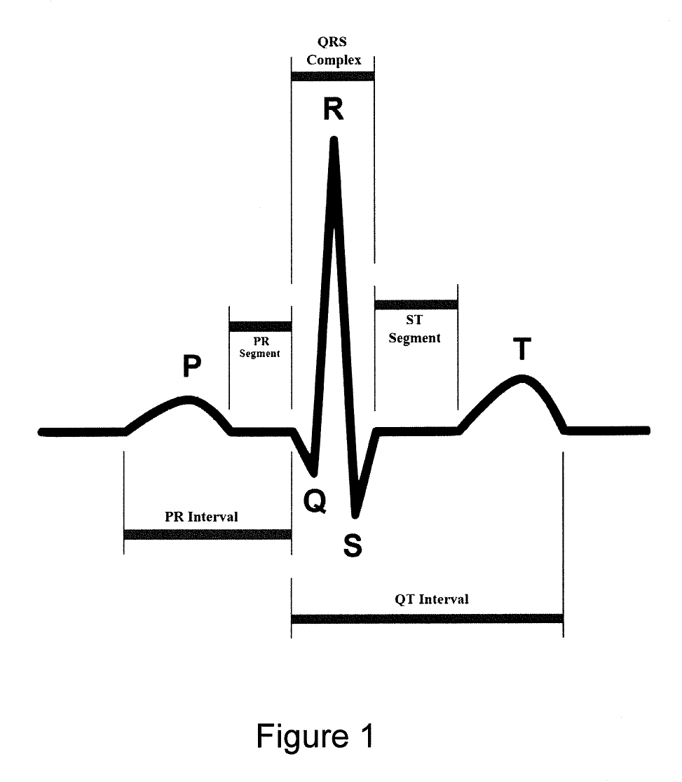

FIG. 1 provides a schematic representation of a normal ECG output, which includes a P, Q, R, S, and T portions (or waves) as is known to those skilled in the art. As is evident from the figure, the R portion is much more pronounced (has a greater amplitude) than the P, Q, S or T portions. Consequently, external factors (e.g., such as movement of the person, environmental factors, posture of the person (e.g., standing)) are much more likely to impact the P, Q, S, and T portions in comparison to the R portion. For example, when a person is engagement in a certain level of activity, the P, Q, S and T portions are much more likely to be corrupted or un-detectable than the R portion. Thus, an ECG test that is performed during a high activity level of the person may provide an inaccurate ECG output, which would lead to an inaccurate diagnosis or assessment. Similarly, environmental factors (e.g., temperature, humidity, etc.) and electrical noise (that may be inadvertently “received” by the ECG sensor system), and other such factors are much more likely to corrupt the P, Q, S and T portions than the R portion.

In one example embodiment, the present invention uses an ECG sensor system and an activity level monitoring system such as an accelerometer. Based on the activity level of the person, portions of the ECG waveform data may be filtered out so as not to provide an inaccurate ECG waveform data. Specifically, the activity level of a person is monitored during collection of ECG waveform data and when the activity level is below a threshold level, the ECG waveform data is output (and processed) including, for example, the P, Q, R, S and T portions. However, when the activity level is above a threshold level, the ECG waveform data is filtered and only a portion of the ECG waveform data is output (and processed) such as only the R portion, which may be used to determine heart rate, etc. Thus, in this example embodiment, the activity level reaching a threshold level acts as a triggering condition that triggers filtering of the ECG waveform data. Other embodiments may use additional or different sensors such as environmental sensors (e.g., temperature, humidity, wind, air pressure, altitude, speed (such as vehicle speed or velocity), underwater depth, GPS location, etc.) and/or other physiological sensors (e.g., measuring posture, body temperature, respiration, skin resistance, breathing rate, etc.) to allow triggering (between filtering and not filtering the ECG waveform data) based on one or more other triggering conditions or events.

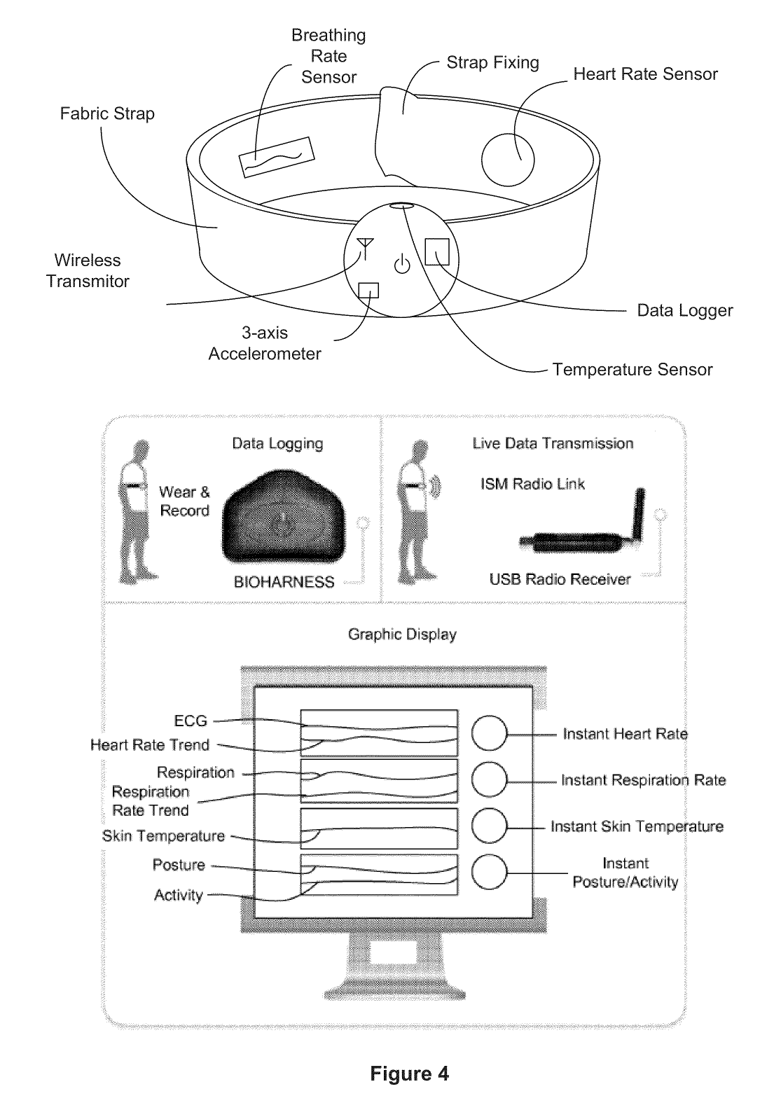

The data used by embodiments of the present invention may be collected and processed by a device such as a BioHarness BT (or the BioHarness or BioHarness HxM), which is commercially available and manufactured by Zephyr Technology of Annapolis, Md. See FIG. 4 which depicts the BioHarness. The BioHarness device measures heart rate, breathing rate, temperature, activity (via an accelerometer), and posture, is battery powered and worn as a chest strap. The BioHarness BT provides ECG waveform data and ECG based measured parameters such as heart rate based on digital signal processing of the R portion to R portion time between beats. They also include a Bluetooth wireless transceiver and internal memory. In other embodiments, the sensor device may be integrated and/or attached to a garment (e.g., shirt). The person may wear the device at home and/or work (or in a clinic environment). The data from the sensors (and in some embodiments, environmental sensors) is regularly collected and stored in memory. Upon collection of ECG and activity level data, the algorithm processes the stored data to determine whether the ECG waveform data should be filtered or not. The algorithm may be executed on the sensor device (e.g., the BioHarness BT) or a computer that receives the data from the sensor device. Alternately, activity level may be measured using an accelerometer such as a tri axial MEMS (micro electronic machine sensor) and ECG waveform data may be provided via a portable ECG device and the data of the ECG filtered according to the principles describe herein.

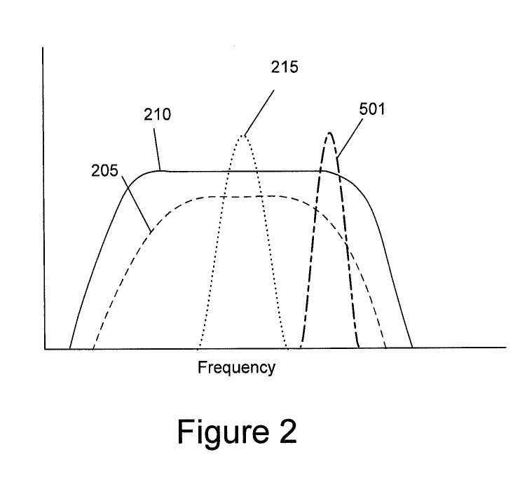

FIG. 2 graphically depicts the frequency band of conventional ECG waveform data represented by dashed line 205 in the frequency domain. A bandpass filter that permits all of the ECG waveform data (the P, Q, R, S, and T portions) to pass through is illustrated by solid line 210. A more narrow filter that allows only the R portion to pass is illustrated by dotted line 215. Such filtering may be performed in hardware (e.g., via analog circuitry), in software (e.g., in a digital signal processor) or some combination of hardware and software. In various embodiments of the present invention, the filtering may be performed in real-time (prior to the data being stored) or at a much later time after collection and storage of the data. Thus, some embodiments of the present invention may include two bandpass filters where one filter is more narrow and filters out all but a subset of frequencies of the wider bandpass filter.

FIG. 2 also shows an interfering signal 501, which in this is caused by the person running. The interfering signal may be determined from the actual signal (i.e., the ECG waveform data) or from another sensor such as an accelerometer. Once the interfering signal is determined, it can be subtracted (or extracted) the collected physiological waveform data to provide the desired physiological waveform data without the interfering signal (noise). This approach may be used for both ECG waveforms and breathing waveforms, where the breathing waveform is extracted from sensors such as from chest expansion.

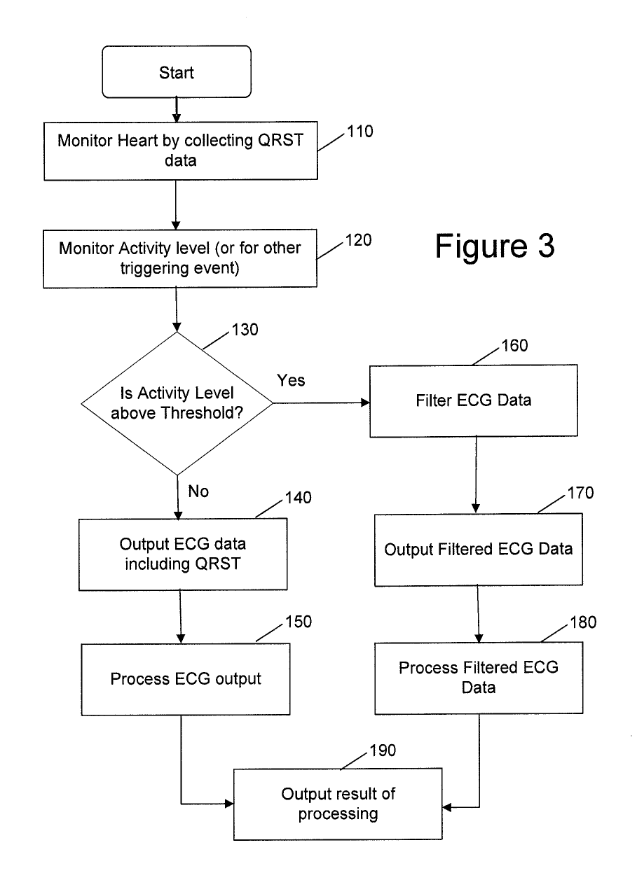

One example algorithm for monitoring the person's heart is described below in conjunction with FIG. 3. The person under test may wear the BioHarness BT or other sensor device(s) to continually (or regularly) collect the person ECG waveform data and activity level data. As discussed, other physiological data and/or other environmental data may be monitored and used to trigger the filtering of ECG waveform data as well, such as signal to noise ratio of the ECG waveform or the person's posture (e.g., measured with the accelerometer). At 110, the person's heart is monitored, capturing the ECG waveform data including collecting entire waveforms and data of the P, Q, R, S, and T portions by the ECG sensor system. The collected ECG waveform data (e.g., that would allow one to graphically depict the waveform such as in FIG. 1) is stored in memory locally (on the person) and/or remotely (via a wireless transmission such as Bluetooth, ANT, and/or a mobile telephone network transmission). At 120, the person's activity level is monitored via the activity level sensor system, which may include an accelerometer. Again, in other embodiments other triggering events may be used. The collected actively level data is stored in memory locally (on the person) and/or remotely (via a wireless transmission such as Bluetooth, ANT, and/or a mobile telephone network transmission). The collection of the ECG waveform data and the activity data (processes 110 and 120) occur concurrently. In addition, the collected ECG waveform data and the activity level data, in some embodiments, may need to be time synchronized at storage or just prior to processing. In other embodiments, the collected ECG waveform data and activity data is not stored (or stored only in non-volatile memory) and step 130 is performed in real time.

The remainder of the processes of FIG. 1 may be performed in real-time or at some later time after collection and storage of the data. At 130, the process determines whether the person's activity level during collection of a set of ECG waveform data is (or was) above a predetermined threshold. The threshold may vary and be based on the humidity, the ECG sensor system, and other factors. If at 130 it is determined that the person's activity level during the collection of the set of ECG waveform data is not above the predetermined threshold, the ECG waveform data is output at 140 (including, for example ECG waveform data or the Q, R, S, and T portions for each heart beat of the set of data) and then processed at 150 via any suitable method for processing normal ECG waveform data (which may include executing a separate and/or specific ECG algorithm). Among other processing results, the processed ECG waveform data may provide the person's heart rate, heart rate recovery, R to R wave timing, R to R wave variability, P wave variability, P to T wave timing, P wave area, P wave width, P wave amplitude, etc.

If at 130 it is determined that the person's activity level during the collection of the set of ECG waveform data is above the predetermined threshold, the ECG waveform data is filtered (e.g., to reject noise) at 160 such as by filtering out the Q, S, and T portions (e.g., as explained with regard to FIG. 2) so that only the R portions of the ECG waveform data remain.

The filtered ECG waveform data is output at 170 and processed at 180. For example, from the filtered ECG waveform data the process may determine the heart rate, the R to R wave timing, and the R to R wave variability.

In addition, Maximum Heart Rate or HRmax may be determined by processing the heart rate to determine the highest heart rate during the activity by performing a moving average (e.g., with a 10 or 15 second trailing window). In addition, Heart Rate Recovery or HRR may also be determined, which is the decrease in heart rate from the time activity stops (Tstop) to a predetermined time (Tlo). In some embodiments of the present invention, the algorithm may compute the HRR using data of the heart rate thirty seconds after the activity stops (i.e., after the activity falls below a threshold) and is computed as the high heart rate (just prior to stoppage of the activity) minus the heart rate thirty seconds after stopping the activity.

Finally, at 190 the processed ECG waveform data (from 150) and the processed filtered ECG waveform data (from 180) are output at 190.

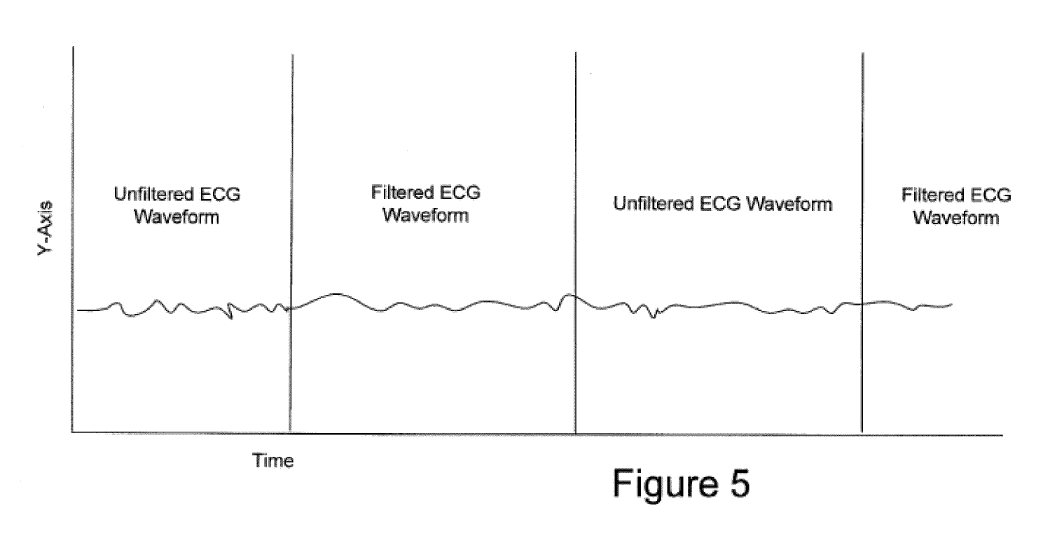

In one embodiment, the output of the unfiltered ECG waveform data (from 140) and the filtered ECG waveform data (from 170 are performed sequentially for each heart beat so that the clinician can view one graphical representation of the person's heart as shown in FIG. 5 in which portions of the ECG waveform data are filtered and other portions are not filtered. Thus, processes 140 and 170 may output their respective data to the same recorder or other destination. In other embodiments, processes 140 and 170 may be omitted so that only the processing results are output. In still other embodiments, processes 140 and 170 may both output their respective data to separate recorders so that there results in two waveforms (one filtered and one not filtered) for the same time period. The collected ECG waveform data may be filtered on a heart beat by heart beat basis or via another suitable interval.

It is worth noting that throughout the collection of data and at various activity levels (both above and below the activity threshold level or other triggering event), the person's heart rate (in this embodiment), R to R timing, R to R variability and other information may readily be determined. In prior art systems, where ECG waveform data may simply be discarded due to inaccuracies caused by high inactivity levels, there would be gaps in the heart rate and other data. In addition, because high activity levels often result in high heart rates, such gaps can be especially critical.

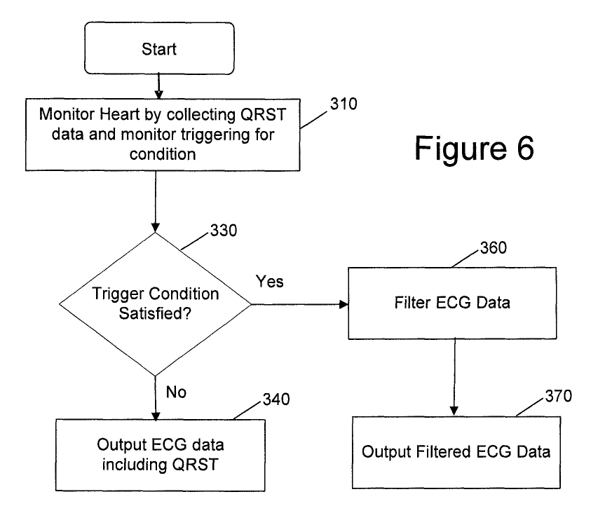

FIG. 6 depicts another example embodiment in which the ECG waveform data is collected and a triggering condition monitored at 310. In this embodiment, a plurality of triggering conditions may be monitored to determine whether the ECG waveform data is filtered or not. For example, conditions that may result in filtering may include one or more of the following: if the activity level (e.g., in VMUs) of the person is above (or below) a threshold, if the person's heart rate is above (or below) a threshold, if the person is lying down (or sitting or running), if the ambient temperature is above (or below) a threshold, if humidity is above (or below) a threshold, if wind speed is above (or below) a threshold, if air pressure is above (or below) a threshold, if altitude is above (or below) a threshold, if vehicle speed is above (or below) a threshold, if water depth (or pressure) is above (or below) a threshold, if body temperature is above (or below) a threshold, if respiration rate is above (or below) a threshold, if GPS location of the person satisfies predetermined criteria (within a geographical area such as at a location), within predetermined time window(s) (e.g., in the morning, afternoon, or evening), etc. In addition, the person may provide a user input that triggers filter or non-filtering of ECG waveform data (such as when a person feels heart palpitations). In addition to any (or all) of the above, signal to noise ratio (SNR) of the ECG waveform below a threshold may be used to trigger filtering. SNR of the ECG waveform data may be computed via any suitable means such as, for example, by dividing the amplitude of the R portion by the root mean square (RMS) voltage (Vrms) of the ECG waveform data of a heart beat (shown in FIG. 1).

The threshold levels of the above conditions to trigger not filtering may be the same or different from the threshold levels to trigger filtering.

In still other embodiments, combinations of any of the above conditions may be used to trigger filtering (and not filtering). For example, the combination of a heart rate above a threshold and a SNR of the ECG waveform above a threshold may be required to not filter the ECG waveform data. As another example, filtering may be triggered only if the activity level is above a threshold and the heart rate is below a threshold.

Referring again to FIG. 6, if the one or more triggering conditions (or combination thereof) are satisfied at 330 the ECG waveform data is filtered at 360 and output at 370. Alternately, if the one or more triggering conditions (or combination thereof) are not satisfied at 330 the ECG waveform data is not filtered is output at 340. The process of FIG. 6 may then repeat for the next ECG waveform data set which may comprise one heart beat or a group of heart beats.

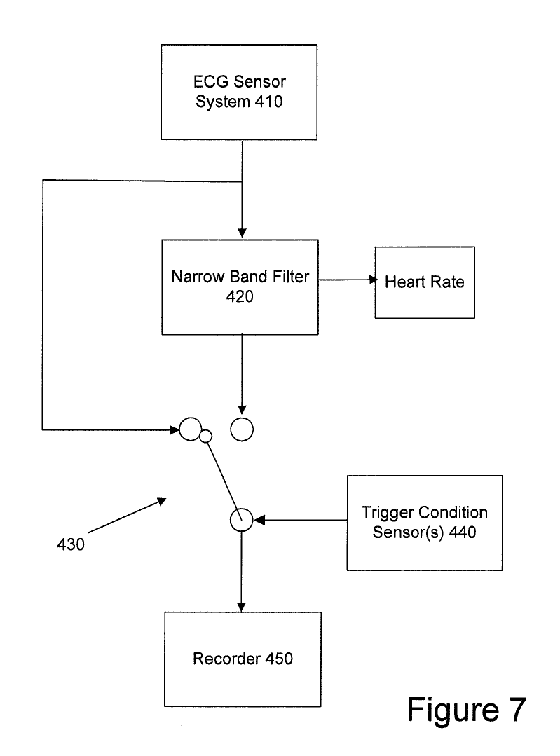

FIG. 7 provides a functional block diagram of an example embodiment of the present invention in which an ECG sensor system 410 provide data to both the narrow band filter 420 and switch 430. The narrow band filter 420 filters the ECG waveform data to provide the R portion of the ECG waveform data to the switch 430 and to output heart rate data. Switch 430 receives a control input from trigger condition sensor(s) 440 (which may be responsive to any of the conditions or triggering events described herein and/or others) operates the switch 430 to provide either unfiltered ECG waveform data or filtered ECG waveform data to the recorder 450 (e.g., the output or stored data). Other functional block diagrams may also be suitable.

Thus, embodiments of the present invention may be used to provide unfiltered ECG waveform data when it less likely to be corrupted (and filtered ECG waveform data at other times) such as when (1) the person is lying or sitting (but filtered when standing); (2) when the person is not running (or not walking); (3) when the person's heart rate is a above a threshold (and more likely to be of interest to the clinician); (4) when the person provides a user input to indicate a user request (indicating the user is feeling palpitations, chest pain, or undergoing some other event); etc.

In some embodiments, instead of filtering the ECG waveform data, no ECG waveform data (filtered or not) is outputted or, alternately measured, unless one or more conditions are satisfied. For example, it may be desirable to only measure and record ECG waveform when the person is under exertion such as when their heart rate is above a threshold. In such an embodiment, the trigger condition sensor 440 may supply its output to actuate the ECG sensor system whose output would be directly supplied to the recorder 450. The method steps may include monitoring one or more trigger conditions, determining whether a trigger condition is satisfied, and collecting and output ECG waveform data if a trigger condition is satisfied.

Algorithms of the present invention can be used while a person is carrying out random events (or exercises) or is performing requested (known) behaviour.

The present invention may be embodied, at least in part, as a computer system (one or more co-located or distributed computers) or cluster executing one or more computer programs stored on a tangible medium. The algorithm may be executed (and computer system located) local or remote from the user. The algorithm may be executed on a computer system that also includes other functions such a telephone or other device (e.g., an IPhone®, IPad®, or Blackberry®), which may have processing and communications capabilities. As discussed, the algorithm may also be stored and executed on the collection device or a separate remote device.

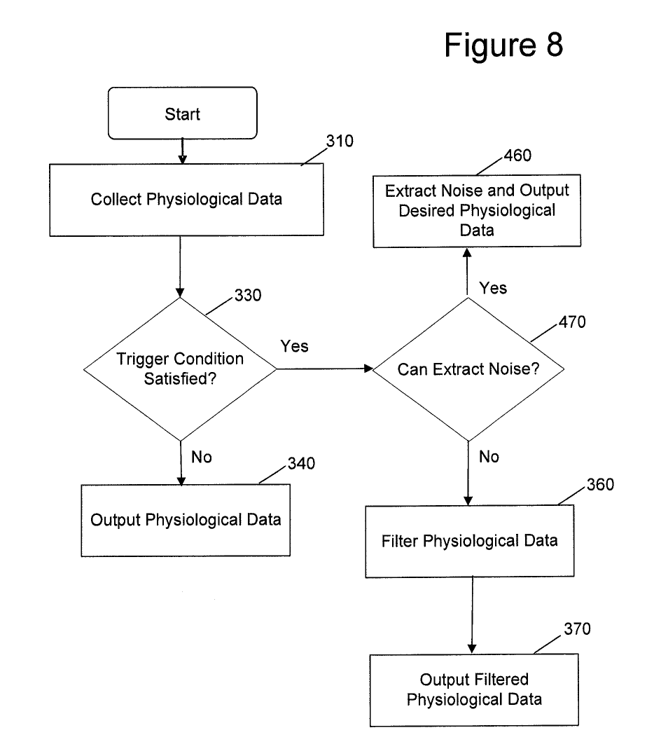

FIG. 8 illustrates yet another example embodiment of the present invention. At 310 the physiological data is collected. At 330, it is determined whether a trigger condition is satisfied which may comprise, for example, determining whether the activity level of the person is above a predetermined threshold and/or whether the signal quality (e.g., SNR) of the collected data is below a threshold. If the trigger condition is not satisfied, the process outputs the physiological data at 340. If the trigger condition is satisfied, it may then be determined whether the noise (i.e., the interfering signal) can be extracted at 470. This question may be determined by information in the physiological data or data from another sensor such as accelerometer (i.e., determining that the user is running). If the noise 501 can be extracted, at 460 the noise data is extracted (or subtracted) from the collected physiological data to provided the desired physiological data and then output (and in some embodiments processed). Thus, for example, a user running may create a large noise spike on a breathing sensor. The system may determine this noise from data from the accelerometer and remove the noise frequency such that the resulting lower frequency smaller amplitude breathing signal is recovered. If the noise signal cannot be extracted, the process continues to 360 where the physiological data is filtered as described above by narrowing the filter to exclude the noise 501 and output at 370. In some embodiments, if the trigger condition is satisfied at 330, the noise is extracted and the desired physiological data (without the nose) is then output (and in some embodiments processed) thereby omitting processes 470, 360, and 370.

Consequently, in one embodiment the method of monitoring the heart of a person, comprises collecting a plurality of sets of ECG waveform data of the person, wherein each set of ECG waveforms, derived numbers such as heart rate and data including a Q portion, an R portion, an S portion, and a T portion for each heart beat and storing the ECG waveform data in a memory. Concurrently with said collecting a plurality of sets of ECG waveform data of the person, the method comprises collecting data of an activity level of the person and storing data the activity level in a memory. The method may further include for each of the plurality of sets of ECG waveform data, determining whether the activity level of the person during collection of a set of ECG waveform data exceeds a threshold; outputting the set of ECG waveform data if the activity level of the person during collection of the set of ECG waveform data does not exceed the threshold; and if the activity level of the person during collection of the set of ECG waveform data exceeds the threshold, filtering the ECG waveform data to provide data of the R portion of the ECG for each heart beat of the set of ECG waveform data and not provide data of the Q portion, S portion, or T portion for each heart beat of the set of data; and outputting the filtered ECG waveform data. The activity of the person during collection of at least one set of ECG waveform data exceeds the threshold, the method may further comprise determining the heart rate of the person over the plurality of the sets of ECG waveform data.

In another embodiment, the method of monitoring the heart of a person may comprise collecting a plurality of sets of ECG waveform data of the person; wherein each set of ECG waveform data includes a Q portion, an R portion, an S portion, and a T portion for each heart beat; storing the ECG waveform data in a memory; concurrently with said collecting a plurality of sets of ECG waveform data of the person, collecting data of an activity level of the person; storing data the activity level in a memory; for each of the plurality of sets of ECG waveform data, determining whether the activity level of the person during collection of a set of ECG waveform data exceeds a threshold; outputting the set of ECG waveform data if the activity level of the person during collection of the set of ECG waveform data does not exceed the threshold; and if the activity level of the person during collection of the set of ECG waveform data exceeds the threshold, filtering the ECG waveform data to exclude the Q portion, S portion, and T portion for each heart beat of the set of data and not excluding the R portion for each heart beat of the set of data; and outputting the filtered ECG waveform data. Wherein activity of the person during collection of at least one set of ECG waveform data exceeds the threshold, the method may further comprise determining the heart rate of the person over the plurality of the sets of ECG waveform data.

In yet another embodiment, the invention may comprise a computer program product, comprising a computer usable medium having a computer readable program code embodied therein, said computer readable program code adapted to be executed to implement a method for monitoring the heart of a person, the method comprising receiving a plurality of sets of ECG waveform data of the person; wherein each set of ECG waveform data includes a Q portion, an R portion, an S portion, and a T portion for each heart beat; storing the ECG waveform data in a memory; receiving data of an activity level of the person collected concurrently with the collection the plurality of sets of ECG waveform data of the person; storing data the activity level in a memory; for each of the plurality of sets of ECG waveform data, determining whether the activity level of the person during collection of a set of ECG waveform data exceeds a threshold; outputting the set of ECG waveform data if the activity level of the person during collection of the set of ECG waveform data does not exceed the threshold; and if the activity level of the person during collection of the set of ECG waveform data exceeds the threshold, filtering the ECG waveform data to exclude the Q portion, S portion, and T portion for each heart beat of the set of data and not excluding the R portion for each heart beat of the set of data; and outputting the filtered ECG waveform data. The activity of the person during collection of at least one set of ECG waveform data exceeds the threshold, the method may further comprise determining the heart rate of the person over the plurality of the sets of ECG waveform data.

In yet another embodiment, the invention may comprise a method of monitoring a vital sign of a person, comprising collecting a plurality of sets of physiological waveform data of the person; for each of the plurality of sets of physiological waveform data, determining whether a trigger condition is satisfied during collection of a set of physiological waveform data; outputting the set of physiological waveform data if the trigger condition is not satisfied during collection of the set of physiological waveform data; and if the trigger condition is satisfied during collection of the set of ECG waveform data, filtering the physiological waveform data and outputting the filtered physiological waveform data.

In yet another embodiment, the invention may comprise a method of monitoring a vital sign of a person, comprising collecting a plurality of sets of physiological waveform data of the person; for each of the plurality of sets of physiological waveform data, determining whether a trigger condition is satisfied during collection of a set of physiological waveform data; outputting the set of physiological waveform data if the trigger condition is not satisfied during collection of the set of physiological waveform data; and if the trigger condition is satisfied during collection of the set of physiological waveform data, performing the steps of: determining whether an interfering signal can be extracted from the set of physiological waveform data and if so, extracting the interfering signal from the physiological waveform data and outputting the physiological waveform data with the interfering signal extracted; if the interfering signal cannot be extracted from the physiological waveform data, filtering the physiological waveform data and outputting the filtered physiological waveform data. The physiological waveform data may comprise breathing waveform data or ECG waveform data. The interference signal may be determined by data from an accelerometer.

In yet another embodiment, the invention may comprise a method of monitoring a vital sign of a person, comprising collecting a plurality of sets of physiological waveform data of the person; for each of the plurality of sets of physiological waveform data, determining whether a trigger condition is satisfied during collection of a set of physiological waveform data; outputting the set of physiological waveform data if the trigger condition is not satisfied during collection of the set of physiological waveform data; and if the trigger condition is satisfied during collection of the set of ECG waveform data, extracting a noise signal from the physiological waveform data and outputting the physiological waveform data with the noise signal extracted. The trigger condition may comprise a SNR below a threshold and/or an activity level above a threshold.

It is to be understood that the foregoing illustrative embodiments have been provided merely for the purpose of explanation and are in no way to be construed as limiting of the invention. Words used herein are words of description and illustration, rather than words of limitation. In addition, the advantages and objectives described herein may not be realized by each and every embodiment practicing the present invention. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention.

What is claimed is:

1. A method of monitoring physiological and motion data of a person, the method comprising:
collecting physiological waveform data of a person comprising at lease one first signal;
collecting motion waveform form data of the person comprising at least one second signal;
using one of the first and second signals as a primary data and the other of the first and second signals as secondary data; and
using the secondary data to determine a filter response of the primary data.

2. The method according to claim 1, further comprising storing the primary and secondary data in a memory.

3. The method according to claim 1, further comprising transmitting the data wirelessly.

4. The method according to claim 1, further comprising transmitting the data by wire.

5. The method according to claim 1, further comprising transmitting the data by optical means or magnetic means.

6. The method according to claim 1, further comprising producing a plurality of sets of primary data and each set of data is filtered a first way is the secondary data is below a threshold and filtered a second way if the secondary data is above a threshold.

7. The method according to claim 6, wherein the threshold condition is satisfied by a combination of two or more sets of data.

8. The method according to claim 6, further comprising outputting the sets of data if the threshold condition is not satisfied during collection of the set of data and if the threshold condition is satisfied during collection of the set of data filtering the data and outputting the filtered data.

9. The method according to claim 6, wherein the threshold comprises one or more selected from the group of: if the person's heart rate is above (or below) a threshold, if the person is lying down (or sitting), if the ambient temperature is above (or below) a threshold, if humidity is above (or below) a threshold, if wind speed is above (or below) a threshold, if air pressure is above (or below) a threshold, if altitude is above (or below) a threshold, if vehicle speed is above (or below) a threshold, if water depth is above (or below) a threshold, if body temperature is above (or below) a threshold, if respiration rate is above (or below) a threshold, if GPS location of the person satisfies predetermined criteria, within predetermined time windows, receiving a user input; and a signal to noise ratio (SNR) of the ECG waveform below a threshold.

10. The method according to claim 1, wherein the second signal comprises at least one of activity, skin resistance, signal noise level, amplitude, respiration, heart rate, humidity, or temperature.

11. The method according to claim 6, wherein the physiological waveform data comprises breathing waveform data.

12. The method according to claim 6, wherein the physiological waveform data comprises ECG waveform data.

13. A method of monitoring the physiological and motion data of a person, comprising:
collecting physiological waveform data of a person;
collecting motion waveform form data of the person;
setting an upper threshold and a lower threshold; and
if either the lower threshold or upper threshold are exceeded by the physiological waveform data or the motion waveform data, transmitting or logging the occurrence.

14. A method of monitoring the physiological and motion data of a person, comprising:
collecting physiological waveform data of a person comprising at lease one first signal;
collecting motion waveform form data of the person comprising at least one second signal;
setting one of the first and second signals as the primary signal and the other of the first and second signals as the secondary signal; and
using the secondary signal to remove artifacts and noise from the primary signal.
